(19) United States
(12) Patent Application Publication
(10) Pub. No.: US 2020/0121565 A1
(43) Pub. Date: Apr. 23, 2020

(54) Modular Partitioned Skin Exfoliant Applicator

(71) Applicant: Sharelle Smith, Redlands, CA (US)

(72) Inventor: Sharelle Smith, Redlands, CA (US)

(21) Appl. No.: 16/164,667

(22) Filed: Oct. 18, 2018

(51) Int. Cl. A61K 8/02; A61Q 19/02; A61Q 19/08

(52) U.S. Cl. CPC A61K 8/0208; A61Q 19/02; A61K 9/703; A61K 8/0212; A61Q 19/08

(57) ABSTRACT

Embodiments are provided that include devices configured to gently exfoliate or remove the upper layers of a user's skin known as the epidermis. The various embodiments provide a skin exfoliant applicator such as a skin-exfoliating face mask that includes multiple modular partitions having varying strengths of exfoliant. These partitions may be applied to different portions of a user's anatomy in a customized configuration. The various embodiments thus enable a customizable skin-exfoliating experience that addresses and accommodates for variations in the epidermis and reduces the likelihood of over exfoliation of sensitive skin.

### BACKGROUND

### 1. Field of the Invention

The present invention relates to cosmetic treatments for temporarily improving the surface texture of human skin. More specifically, the present invention relates to treatments involving the removal of, or that promote the removal of, outer layers of the epidermis by use of a modular partitioned face mask.

### 2. Background Information

The cosmetics industry has utilized scientific breakthroughs to develop an ever-expanding variety of skin care products. These products provide benefits including anti-aging, hydration, dermal nourishment and growth, oil reduction, and cleanser amongst others. Many of these products are applied topically and act on the top two layers of the facial derma known as the epidermis. However, proper penetration of topical skin treatments into the top layers of the facial epidermis requires that the skin be clean. Clean skin is relatively free of oils, debris, and dead skin, which may act as a barrier between the derma and topical skin treatments.

Oil, debris, and dead skin may be removed from the surface of the epidermis by way of one or more methods of exfoliation. Skin may be exfoliated mechanically or chemically. Mechanical exfoliation involves rubbing, abrading, or scrubbing the skin with microfiber cloths, adhesive exfoliation sheets, micro-bead facial scrubs, crepe paper, crushed apricot kernel or almond shells, sugar or salt crystals, pumice, and abrasive materials such as sponges, loofahs, or brushes, Chemical, Chemical exfoliants include scrubs containing salicylic acid, glycolic acid, fruit enzymes, citric acid, or malic acid which may be applied in high concentrations by a medical professional, or in lower concentrations in over-the-counter products. Chemical exfoliation may involve the use of products that contain alpha hydroxy acids (AHAs), beta hydroxy acids (BHAs), or enzymes that act to loosen the glue-like substance that holds the cells together, allowing them to ease away.

Standard techniques, both mechanical and chemical suffer from the drawback of treating the epidermis in a uniform manner. However, many people have facial epidermis that differs in texture and type across regions of the face, making uniform exfoliation methods less than wholly successful. Further, facial skin changes in type and texture as a person ages, increasing the complexity of facial epidermis variation. Thus, uniform. exfoliation methods do not adequately address these variations in facial epidermis.

A solution is needed that provides gentle epidermal exfoliation that is customizable to the variations in individual skin type. The various embodiments provide a modular partitioned exfoliating face mask that may be easily customizable to address the skin type of each portion of a user's skin.

### SUMMARY OF THE INVENTION

The above described problems are solved and a technical advance achieved by the modular partitioned skin exfoliant applicator for exfoliating and cleansing the epidermis by providing anatomy-specific partitions of varying exfoliant strength, which are modular in nature and may be customized to meet the needs of a user's skin type. The skin-exfoliating mask thus enables users to configure a skin exfoliation treatment that accounts for the difference in skin type across a user's anatomy and throughout the life cycle of the user's skin. This advantage may reduce the likelihood of exposing the epidermis to skin exfoliants of uniform strength and concentration, which may damage sensitive portions of a user's epidermis.

For example, an embodiment modular partitioned face mask may include multiple partitions, each of the multiple partitions having a skin-exfoliating adhesive coating. In another example, an embodiment modular partitioned exfoliant applicator may include multiple partitions configured to exfoliate the epidermis of a user's skin when applied thereto. In a further example, an embodiment modular partitioned face mask may include multiple partitions shaped to fit a different portion of a user's facial topography, each of the multiple partitions having a skin-exfoliating adhesive coating, in which the skin-exfoliating adhesive coating of each of the multiple partitions is of a strength corresponding to a portion of a user's facial topography or a characteristic of the user's epidermis.

The details of one or more embodiments are set forth in the accompanying drawings and the description below. Other features, objects, and advantages of the invention will be apparent from the description and drawings, and from the claims.

### BRIEF DESCRIPTION OF THE DRAWINGS

The accompanying drawings, which are incorporated herein and constitute part of this specification, illustrate exemplary embodiment of the invention, and together with the general description given above and the detailed description given below, serve to explain the features of the invention.

FIG. 1 is a diagram of an example configuration of partitions of a modular partitioned skin exfoliant applicator as a skin-exfoliating face mask applied to a user's face according to the various embodiments.

FIG. 2 is a chest partition of the face mask according to the various embodiments.

FIG. 3 is a neck partition of the face mask according to the various embodiments.

FIG. 4 is right and left eye partitions of the face mask according to the various embodiments.

FIG. 5 is right and left cheek partitions of the face mask according to the various embodiments.

FIG. 6 is a forehead partition of the face mask according to the various embodiments.

FIG. 7 is a nose partition of the face mask according to the various embodiments.

FIG. 8 is a mouth partition of the face mask according to the various embodiments.

FIG. 9 is right and left chin partitions of the face mask according to the various embodiments.

FIG. 10 is a patch partition of the face mask according to the various embodiments.

FIG. 11 is a diagram illustrating the partition material and exfoliant layer of the partitions of the modular partitioned skin exfoliant applicator according to the various embodiments.

FIG. 12 is a cross-section diagram of a partition of the modular partitioned skin exfoliant applicator according to the various embodiments.

### DETAILED DESCRIPTION

The various embodiments will be described in detail with reference to the accompanying drawings. Wherever possible, the same reference numbers will be used throughout the drawings to refer to the same or like parts. References made to particular examples and implementations are for illustrative purposes, and are not intended to limit the scope of the invention or the claims.

In overview, the various embodiments include devices configured to gently exfoliate or remove the upper layers of a user's skin known as the epidermis. Regular exfoliation of the epidermis is an essential part of encouraging and maintaining proper skin health. Standard exfoliation techniques provide uniform application of a mechanical or chemical exfoliant to the skin without regard for variations in skin type, age, or sensitivity throughout the epidermis. Such uniform application of exfoliant can lead to under exfoliation of tough or thick skin as well as over exfoliation or abrasion of sensitive or thin skin. The various embodiments disclosed herein provide a skin exfoliant applicator such as a skin-exfoliating face mask that includes multiple modular, customizable partitions including differing strengths of exfoliant, which may be applied to different portions of a user's anatomy. The various embodiments thus enable a customizable skin-exfoliating experience for users that addresses and accommodates for variations in the epidermis and reduces the likelihood of over exfoliation of sensitive skin.

In the various embodiments, the modular, partitioned, skin-exfoliating mask may include multiple partitions, each of the multiple partitions having a skin-exfoliating adhesive coating. One side of each partition may be coated in an adhesive material suitable for exfoliating the epidermis when applied to and subsequently peeled away from a user's skin. Thus, in various implementations may include a modular, partitioned skin exfoliant applicator in which the multiple partitions are configured to exfoliate the epidermis of a user's skin when applied thereto. The multiple partitions may be shaped to conform to different portions of a user's anatomy to ensure contact between the partition and the surface area of the user's skin. Further, each of the skin exfoliant of each of the partitions may be of a strength that is appropriate for the region of the user's anatomy to which the partition is configured to conform. For example, a nose partition may have a “nose” shape and may have an adhesive exfoliant coating of a strength that is suitable for the sensitive skin around the nose.

Throughout daily life, the epidermis may collect oil, debris, and dead skin, making it difficult for new skin cells lying under the epidermis to breathe and grow. Further, clogged or dirty pores in the epidermis may also reduce the efficacy of skin care treatments by preventing the treatments from permeating throughout the epidermis and into deeper layers of the user's skin. During exfoliation, the upper layers of skin, known as the epidermis are removed, taking dirt, dead skin, and oil accumulation with it. This leaves the underlying, healthier skin cells exposed as the new surface of the skin. If done properly, exfoliation is an important component of the skin rejuvenation cycle. However, if the skin is over exfoliated, too much skin may be removed, damaging underlying skin cells that are still forming If skin is underexfoliated, then patches of dead skin may remain, leading to uneven exposure of new skin cells on the surface and uneven permeation of topical skin treatments.

The various embodiments may reduce the likelihood of overexfoliation and underexfoliation by providing modular partitions of a skin exfoliant applicator. The individual partitions contain skin exfoliant appropriate for the skin type of specific parts of a user's anatomy. For example, in various embodiments, each of the multiple partitions may be shaped to fit a different portion of a user's facial topography. In other embodiments, each of the multiple partitions may be shaped to fit a different portion of a user's anatomy such as a foot, hand, or other portion of the user's body. Each of the multiple partitions may be configured to conform to the contours of a different portion of a user's facial topography or a portion of the user's anatomy.

The skin exfoliant of each partition may vary in strength and composition. The strength and composition may be of a type that is suitable for skin of a certain age and/or for the type of skin located near a part of human anatomy. For example, skin around the eyes tends to be thin and delicate by comparison to the skin of the forehead region, making a weak exfoliant more effective on the eye region than the forehead region. A user's age may also impact the characteristics of their skin. As a person ages, the dermis thins and regenerates more slowly. The cheek skin of a 25-year old may be thicker and less sensitive than that of a 72-year old, but may have less dead skin accumulation. Thus, a strong but gentle exfoliant may be required for the 72-year old, while a weaker exfoliant may be effective for the 25-year old. To address these variations in skin type and exfoliation requirements, the multiple partitions may each include a skin exfoliant appropriate for an age range of user (e.g., 30-40) and/or a body part for which the partition is to be applied.

The type of skin exfoliant included in each partition may vary and may include a chemical exfoliant. Optionally, the skin exfoliant may also include a mechanical exfoliating element such as trace amounts of sand, sugar, pumice, or the like. The various embodiments include partitions with skin exfoliant that is well suited for skin of a certain age and/or of a certain body part. In some implementations, the exfoliant may be a skin-exfoliating adhesive coating on one side of each partition. The skin-exfoliating adhesive coating of each of the multiple partitions may be of a strength corresponding to a portion of a user's facial topography or to a portion of a user's anatomical topography, e.g., fingers, toes, elbows, shins, etc. Each partition may be applied to a user's skin by placing the skin-exfoliating adhesive coating in contact with the skin. Pressure may be applied to the exposed side of the partition to improve the connection between the skin-exfoliating adhesive coating and the user's epidermis. The skin-exfoliant adhesive coating may attract oils, debris, and dead skin lying on the epidermis surface. The partition may then be peeled away or otherwise removed from the user's skin to exfoliate and remove oils, debris, and dead skin.

To improve user experience and ease of use, in various embodiments the strength of the skin-exfoliating adhesive coating of each of the multiple partitions may be indicated by visual indicia on each of the multiple partitions. Color coding of partitions may be used to provide users with ease reference as to which partitions should be applied to skin of a characteristics. For example, partitions intended for older, sensitive, or acne-impacted skin may be marked with a particular color or pattern. In some implementations, partitions intended for common regions of the human anatomy may be marked with visual indicia such as a pattern. For example, partitions intended for the neck and décolletage may be marked with stripes. Stars, or a checked pattern to indicate that these partitions are intended for use in a common region of the user anatomy and may have a skin-exfoliant of similar strength.

In various embodiments, the multiple partitions may be made of the same base material or may differ slightly in composition so as to conform to the anatomy to which the partition is applied. Various thin film plastics may be well suited for use as the base material for each partition because these materials are flexible in nature and easily conform to the underlying topography of an object to which they are applied. However, cloth and mesh materials may also be used as the base material for the multiple partitions of the skin exfoliant applicator. For example, materials such as high quality latex, flexible cotton, thin plastics, and woven fabrics may be used in construction of the partitions.

In practice, a user may select a partition suitable for each specific portion of the face (or other anatomy) for which exfoliation treatment is desired. Each partition may be selected based on the suitability of the skin exfoliant for the skin type, age, and sensitivity of the portion of the face to which the partition will be applied. Partitions having the desired strength and type of skin exfoliant may be easily identified by a user via the visual indicia of the partitions. Once the user has selected all applicable partitions, a protective backing or seal may be peeled away to expose the exfoliating adhesive coating on a partition. The side of the partition including the exfoliant adhesive coating is then applied directly to the surface of the skin, and the partition smoothed into place so that it conforms to the shape of the underlying anatomy. An application period or wait period, during which the partitions are left in contact with the skin may vary from user to user based on skin type and condition. The partition may then be peeled away from the skin to remove excess oil, dead skin, and debris. This process is repeated with all partitions. In some embodiments, all partitions may be applied to the surface of the skin and then removed one at a time. In other embodiments, partitions may be used individually and removed prior to use of additional partitions.

Users may repeat the application and removal of one or more of the partitions to further improve exfoliation and cleansing of the skin. The user may repeat the procedure with new partition pieces for all or some of the same portions of the body recently treated. For example, if the user applied partitions to all portions of the face and neck, the user may apply a second round of partitions over some or all portions of the face. Repeating the application and removal of the partitioned exfoliant applicator may improve overall exfoliation of treated areas. Multiple treatments may be particularly useful for persons who wear makeup, have skin that overproduces oils, or is damaged by exposure to the environment, because this skin may have additional layers of dead skin, dirt or debris accumulated on the epidermal surface.

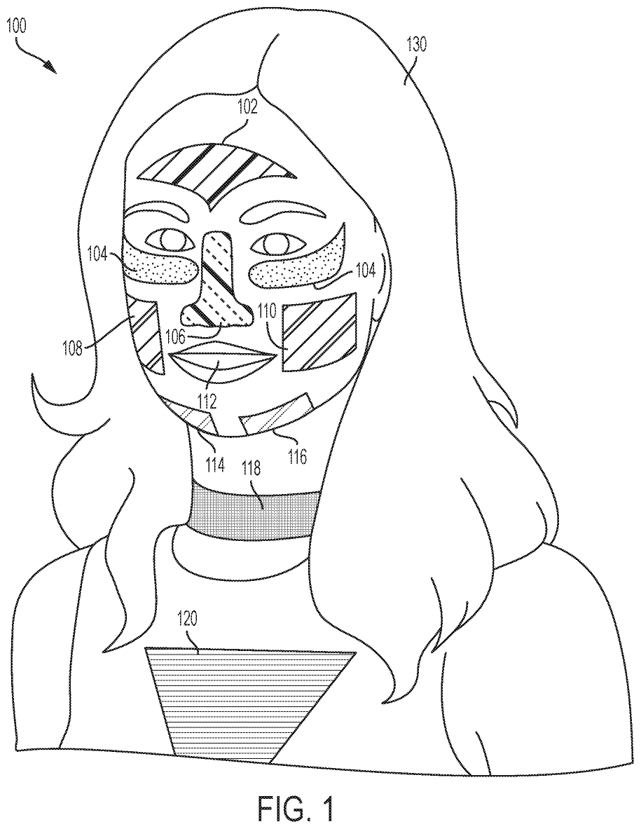

The various embodiments thus provide a modular partitioned skin-exfoliant applicator such as a face mask, which has modular partitions that may be swapped out into a configuration that is customized for each user. The user may select each partition or set of partitions according to the desire strength and character of the skin exfoliant for the user anatomy to which the partitions will be applied. Users may identify the desired skin exfoliant strength and character by means of one or more visual indicia such as color, pattern, or surface texture. The selected partitions may then be applied to proscribed portions of a user's anatomy to collect and remove oil, debris, and dead skin from the epidermis, an example of which is illustrated in FIG. 1.

FIG. 1 illustrates an example modular partitioned skin exfoliant applicator as a skin-exfoliating face mask 100, i.e. a face mask. The face mask 100 includes a number of partitions 102, 104-120 applied to the face of a user 103. Each of the partitions 102, 104-120 may have a shape and size such that the partition is configured to conform to the shape and surface of the underlying portion of face of the user 103. As each partition 102, 104-120 is removed from the surface of the face, the partition surface in contact with the skin of the user 103 along with any dead skin, oils, and debris attached thereto, will detach from the surface of the face. After removal of the face mask 100 clean, fresh skin is exposed and ready for application of any additional cosmetic treatments.

The example face mask 100 has multiple partitions 102, 104-120, which may be applied to the surface of the skin of a user 103. As shown, the face mask 100 is not a single piece of material covering the user's face. Rather, the multiple partitions 102, 104-120 may be available in different sizes (e.g., multiple sizes of forehead partition 102), enabling users to select partitions that are appropriately sized to their face.

Further, the exterior surface of each partition may include visual indicia such as distinct color, pattern, or visible surface texture. In various embodiments, visual indicia provide information about the strength and type of skin exfoliant included in a particular partition. For example, partitions having an external surface with a diagonally striped pattern, such as forehead partition 102, nose partition 106, and cheek partitions 108, may include a skin exfoliant suitable for use with oily skin. The appearance of dark stripes, small stripes, and dashed stripes may indicate a strength of the skin-exfoliant, e.g., dashed stripes for sensitive or young skin, dark lines for skin of average sensitivity, and light kinds for older or tougher skin. The inclusion of visual indicia on each partition enables users and/or their physicians to easily select partitions having a skin exfoliant type and strength that is appropriate for each portion of the user's anatomy intended for treatment. The different partitions are thus modular and customizable, to provide users with a skin exfoliant applicator that can be tailored to the exfoliation needs of individual portions of the user's anatomy.

Each of the partitions 102, 104-120 may include one or more layers of skin exfoliant. When placed in contact with the skin of a user 103, the exfoliant may adhere to the surface of the skin, including any dead skin, oil, and debris laying on the surface of the skin. Dead skin, oils, and debris re not integral to the epidermis. Rather, these particles sit loosely on the surface of the skin, where they may clog pores, causing acne, or block the flow of oxygen to the underlying live skin. In the various embodiments, the one or more layers of skin-exfoliant may attract or adhere to the loose particles and remove them from the surface of the skin when the partition is detached.

The partitions 102, 104-120 are applied directly to the skin of the corresponding portion of the user's anatomy. For example, the side of a partition 102, 104-120 bearing the skin exfoliant may be exposed and pressed down into contact with the skin surface, using the fingers, a roller, or a firm object. The partition 102, 104-120 may be smoothed down to improve connection between the skin exfoliant and the surface of the user's skin. Pressing the partitions 102, 104-120 down and smoothing them out may reduce pocketing (air trapped underneath the partition that prevents contact between the skin and the exfoliant) and consequently reduce the likelihood that portions of the skin surface will remain untreated by skin exfoliant.

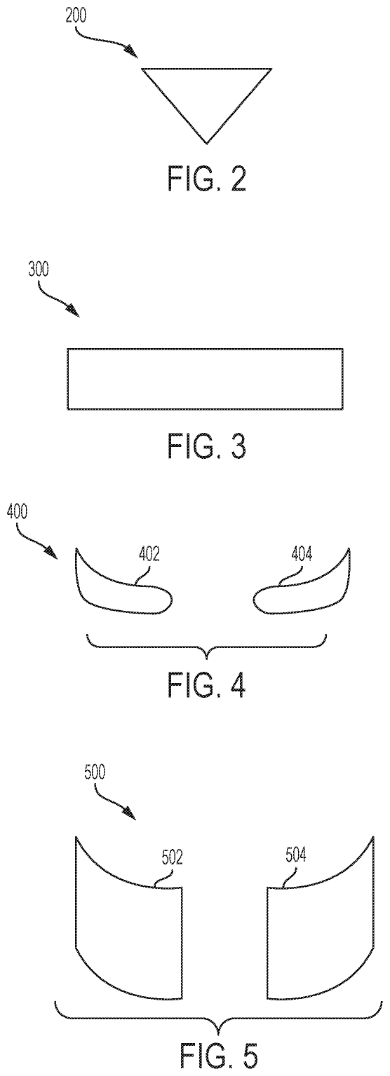

In addition to the skin-exfoliating face mask 100 discussed above, the various embodiments (including, but not limited to, embodiments discussed below with respect to FIGS. 2-12) may be implemented in a wide variety of partition configurations, which may include multiple partitions, partitions configured to fit multiple body parts, or any combination thereof.

FIGS. 2-10 illustrate exemplary partitions of a modular partitioned skin exfoliant applicator. The partitions illustrated in FIGS. 2-10 may correspond to those shown in FIG. 1 and may be configured for application to portions of a user's face. The size and shape of the illustrated partitions is for exemplary purposes and is not to scale or precise in boundaries. The partitions are illustrated without visual indicia, however examples of the visual indicia as applied to the various partitions may be found in detail with reference to FIG. 1.

FIG. 2 illustrates example solutions for a chest partition of a modular partitioned skin exfoliant applicator in the form of a face mask (e.g., face mask 100). With reference to FIGS. 1-2, chest partition 200 may correspond to chest partition 120 applied to the chest of a user 103 in FIG. 1. The chest partition 200 may have a generally triangular or trapezoidal shape configured to conform to the décolletage of a user 103. Because some users may have a larger décolletage area than others, the size and shape of the chest partition 200 may vary. Optionally, chest partition 200 may include a stronger skin exfoliant than some other partitions, because the décolletage skin may be less sensitive than the skin of the facial areas. The chest partition 200 may be applied to the user's chest such that the widest lateral edge is closest to the user's neck and the form tapers as it extends downward.

FIG. 3 illustrates example solutions for a neck partition of a modular partitioned skin exfoliant applicator in the form of a face mask (e.g., face mask 100). With reference to FIGS. 1-3, neck partition 300 may extend around the front and sides of the user's neck when applied. The neck partition 300 may have a rectangular or generally parallelepoids shape. When applied to the neck of a user, the neck partition 300 may have the appearance of a “choker” necklace. The surface of the neck partition 300 applied to the user's skin may gently exfoliate the skin along the front and sides of the neck. Optionally, the neck partition 300 may be sized so as to extend all the way around the user's neck and cover the rear of the neck as well. Like the décolletage, neck skin may be tough and may require a stronger exfoliant than partitions intended for application to a user's face.

FIG. 4 illustrates example solutions for eye partitions of a modular partitioned skin exfoliant applicator in the form of a face mask (e.g., face mask 100). With reference to FIGS. 1-4, eye partitions 400 may include a left eye partition 402 and a right eye partition 404. Each of left eye partition 402 and right eye partition 404 may be applied individually to the portion of the face lying directly underneath corresponding eyes. The skin of the area surrounding the eye is delicate and thin Overexfoliation of the area surrounding the eye area can lead to red, raw, and even painful skin irritation. The use of a strong exfoliant along the eye area may be inappropriate or dangerous for many users. Thus, the modular eye partitions 400 of the face mask 100 may enable users to select a gentle exfoliant that will not overexfoliate the sensitive skin of the region below their eyes.

In various embodiments, the left eye partition 402 and the right eye partition 404 may have a generally ovular or elliptical shape so as to conform to the shape of the portion of the face lying directly under and to the side of a user's eyes. Optionally, the eye partitions 400 may have a “cat eye” wing on the outer ends of the partition. This wing may cover the delicate skin lying below and extending outwardly past the immediate eye area and may thus improve overall coverage of the skin exfoliant along the region below and to the outside of the eye.

FIG. 5 illustrates example solutions for cheek partitions of a modular partitioned skin exfoliant applicator in the form of a face mask (e.g., face mask 100). With reference to FIGS. 1-5, cheek partitions 500 may include left cheek partition 502 and right cheek partition 504. The cheek partitions 502 may be configured to cover and conform to the portion of the user's face lying between the ears and the nose, and the eyes and jawbone. The edges of the cheek partitions 500 may be curved to mimic the contour of human cheeks. As shown in FIG. 1, the cheek partitions 500, i.e., 108, 110, may be separate and distinct from the eye partitions 400, i.e., 104.

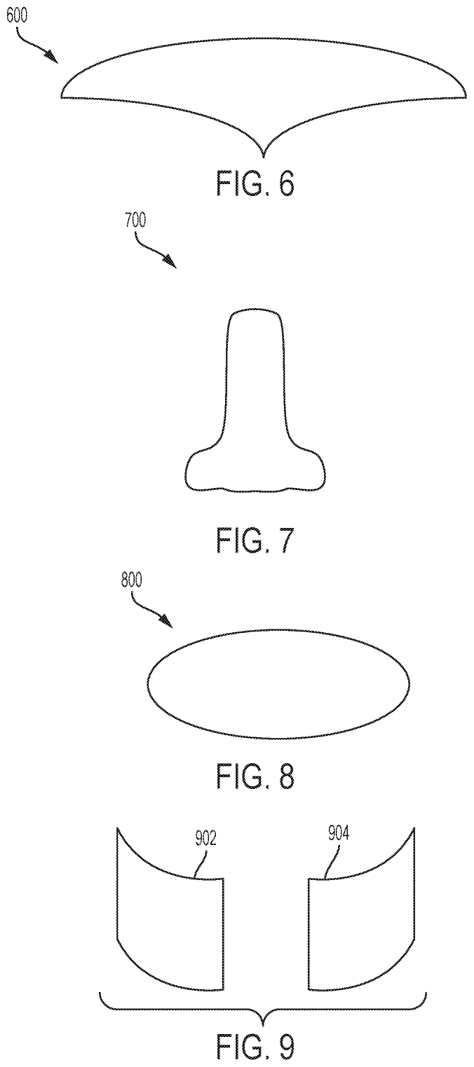

FIG. 6 illustrates example solutions for a forehead partition of a modular partitioned skin exfoliant applicator in the form of a face mask (e.g., face mask 100). With reference to FIGS. 1-6, forehead partition 600 may be a large partition configured to cover and conform to the forehead. The upper edge of the forehead partition 600 may meet a user's hairline when applied to the face. Alternatively, the upper edge of the forehead partition 600 may rest just below the hairline. The lower edges of the forehead partition 600 may have concave curvature to follow the curve of a user's brow bones. The lower edges may meet in a lower apex. In some embodiments, the lower apex of the forehead partition 600 may overlap or meet the top of the nose partition (e.g., 106).

FIG. 7 illustrates example solutions for a nose partition of a modular partitioned skin exfoliant applicator in the form of a face mask (e.g., face mask 100). With reference to FIGS. 1-7, nose partition 700 may have a generally triangular shape. In various embodiments, the nose partition 700 may have an upper portion which is elongated, tubular, or rectangular in shape. The nose partition 700 may have two side portions, which protrude along opposing sides of the bottom of the partition. Optionally, the nose portion 700 may just have a triangular shape. Because the human nose may have a variety of shapes, the nose partition 700 may have many variations in shape. The size and shape must be sufficient to cover the surface area of the user's nose.

FIG. 8 illustrates example solutions for a mouth partition of a modular partitioned skin exfoliant applicator in the form of a face mask (e.g., face mask 100). With reference to FIGS. 1-8, mouth partition 800 may be used to gently exfoliate the lips and surrounding skin. The skin of the human mouth is generally delicate and should not be exposed to harsh exfoliants. Further, exfoliating the mouth exposes the user to possible ingestion of harmful chemicals. Because the mouth partition 800 of the various embodiments is separate and distinct from other partitions, the skin exfoliant of the mouth partition 800 may optionally be of a different composition than that of the other partitions.

FIG. 9 illustrates example solutions for a chin partitions of a modular partitioned skin exfoliant applicator in the form of a face mask (e.g., face mask 100). With reference to FIGS. 1-9, chin partitions 900 may include both a left chin partition 902 and right chin partition 904. The chin partitions 900 may have a shape similar to the cheek partitions 500 described in greater detail with reference to FIG. 5. However, as illustrated in FIG. 1, the chin partitions 900, i.e., partitions 114, 116, may be shorter in height and/or smaller in overall dimension than the cheek partitions 500, i.e., 108, 1110. The chin partitions 900 may be configured to cover and conform to the areas lying along the jawbone and over part of the chin of a user's face.

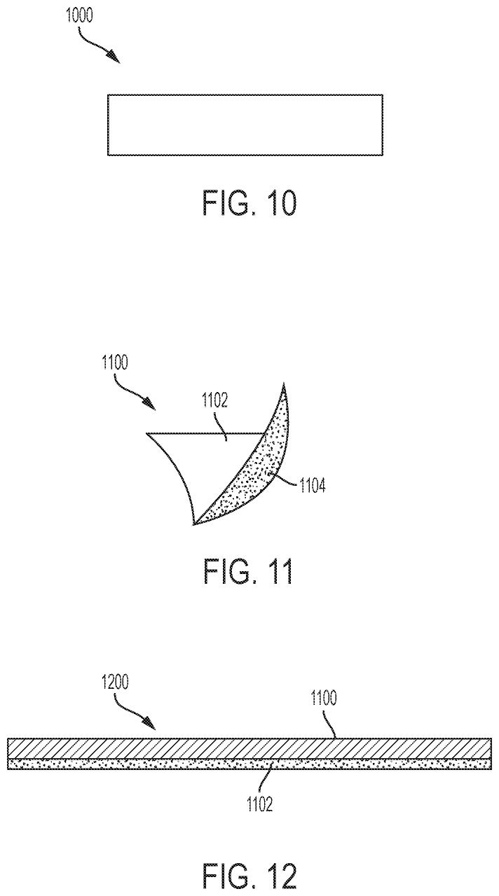

FIG. 10 illustrates example solutions for a patch partition of a modular partitioned skin exfoliant applicator in the form of a face mask (e.g., face mask 100). With reference to FIGS. 1-10, patch partition 1000 may have a variety of shapes such as a rectangular, square, trapezoidal, or circular shape. The patch partition 1000 is not configured to conform to a particular portion of the human anatomy. Rather, the path partition 1000 may be placed anywhere on the body of a user 103. Such versatile partitions may be useful in applying skin exfoliant to areas of the body that are not adequately covered by specialized partitions such as those described with reference to FIGS. 2-9. Patch partition 100 may also be well suited for applying skin exfoliant to portions of the human anatomy without a standard shape.

FIGS. 11 and 12 illustrate exemplary bi-layer partitions of a modular partitioned skin exfoliant applicator. The partitions illustrated in FIGS. 11 and 12 may correspond to those shown in FIGS. 2-10. The illustrated partitions include a bi-layer structure, however additional layers are contemplated and may be added to improve the strength or efficacy of the skin-exfoliant or adhesive.

FIG. 11 illustrates example solutions for a bi-layer partition of a modular partitioned skin exfoliant applicator such as face mask 100. With reference to FIGS. 1-11, partition 1100 may have a backing layer 1102 and an exfoliant layer 1104. In various embodiments, the exfoliant layer 1104 may be a skin adhesive suitable for temporarily adhering the backing layer 1102 to the skin of a user 103 upon contact and the exertion of light pressure. In various embodiments, the visual indicia may be printed upon the backing layer 1102, which is the outer layer and as such is visible to a user 103 throughout the application process.

The backing layer 1102 may be constructed of a variety of materials such as plastic (e.g., thin films, ultra-light plastics), high quality latex, flexible cotton, woven fabric, vinyl, or other lightweight flexible material. In some embodiments, different backing layer materials may be used to construct different partitions. Larger partitions such as the chest partition 200 and the forehead partition 600 may be constructed of thicker or less flexible materials, while the nose partition 700 and the eye partitions 400 may be constructed of very thin, light-weight materials that easily conform to the shape of the underlying surface. In some embodiments, all partitions may be constructed of the same backing layer material.

FIG. 12 illustrates example solutions for a bi-layer partition of a modular partitioned skin exfoliant applicator such as face mask 100. With reference to FIGS. 1-12, partition 1200 is a cross-sectional view illustrating the backing layer 1102 and an exfoliant layer 1104 discussed in detail with reference to FIG. 11. As illustrated in FIG. 12, the exfoliant layer 1104 may be the same as the adhesive coating. Specifically, the exfoliant layer may contain both the skin-exfoliant and a skin adhesive. Such embodiments may include a chemical skin exfoliant and skin adhesive combination. The exfoliant layer may include one more combinations of acrylic, epoxy resin, or rubber-based, polyurethane, isocyanate, acrylate, and vinyl resin adhesives and a mechanical or chemical exfoliant. In some embodiments, the adhesive coating layer may be distinct from the exfoliant layer 1104. For example, the adhesive coating may be applied to the backing layer 1102 prior to application of the exfoliant layer 1104 or vice versa.

The foregoing descriptions and the diagrams are provided merely as illustrative examples and are not intended to require or imply that the various embodiments must be configured or performed in the order presented. As will be appreciated by one of skill in the art the order of blocks in the foregoing embodiments may be performed in any order. Words such as “thereafter,” “then,” “next,” etc. are not intended to limit the order or arrangement; these words are simply used to guide the reader through the description of the methods. Further, any reference to claim elements in the singular, for example, using the articles “a,” “an” or “the” is not to be construed as limiting the element to the singular.

The preceding description of the disclosed embodiments is provided to enable any person skilled in the art to make or use the present invention. Various modifications to these embodiments will be readily apparent to those skilled in the art, and the generic principles defined herein may be applied to other embodiments without departing from the spirit or scope of the invention. Thus, the present invention is not intended to be limited to the embodiments shown herein but is to be accorded the widest scope consistent with the following claims and the principles and novel features disclosed herein.

What is claimed is:

1. A modular partitioned face mask, comprising:
multiple partitions, each of the multiple partitions having a skin-exfoliating adhesive coating.

2. The modular partitioned face mask of claim 1, wherein each of the multiple partitions is shaped to fit a different portion of a user's facial topography.

3. The modular partitioned face mask of claim 2, wherein each of the multiple partitions is configured to conform to the contour of a different portion of a user's facial topography.

4. The modular partitioned face mask of claim 1, wherein the skin-exfoliating adhesive coating of each of the multiple partitions is of a strength corresponding to a portion of a user's facial topography.

5. The modular partitioned face mask of claim 4, wherein the strength of the skin-exfoliating adhesive coating of each of the multiple partitions is indicated by visual indicia on each of the multiple partitions.

6. The modular partitioned face mask of claim 5, wherein the visual indicia is a color or pattern.

7. The modular partitioned face mask of claim 1, wherein the skin-exfoliating adhesive coating of each of the multiple partitions is of a strength corresponding to a characteristic of a user's epidermis.

8. The modular partitioned face mask of claim 1, wherein the multiple partitions are constructed from at least one of flexible cotton, latex, plastic, or fabric.

9. The modular partitioned face mask of claim 1, wherein the multiple partitions comprise two or more of: a chest partition, a neck partition, eye partitions, cheek partitions, a forehead partition, a nose partition, a mouth partition, and chin partitions.

10. A modular partitioned exfoliant applicator, comprising:
multiple partitions configured to exfoliate the epidermis of a user's skin when applied thereto.

11. The modular partitioned exfoliant applicator of claim 10, wherein each of the multiple partitions is shaped to fit a different portion of a user's anatomy.

12. The modular partitioned exfoliant applicator of claim 11, wherein each of the multiple partitions is configured to conform to the contour of a different portion of a user's anatomy.

13. The modular partitioned exfoliant applicator of claim 10, wherein a strength of a skin-exfoliant of each of the multiple partitions corresponds to a portion of a user's anatomical topography.

14. The modular partitioned exfoliant applicator of claim 13, wherein the strength of the skin-exfoliant of each of the multiple partitions is indicated by visual indicia on each of the multiple partitions.

15. The modular partitioned skin applicator of claim 14, wherein the visual indicia is a color or pattern.

16. The modular partitioned exfoliant applicator of claim 10, wherein a strength of a skin-exfoliant of each of the multiple partitions corresponds to a portion of a characteristic of the user's epidermis.

17. The modular partitioned skin applicator of claim 10, wherein the multiple partitions are constructed from at least one of flexible cotton, latex, plastic, or fabric.

18. The modular partitioned skin applicator of claim 10, wherein the multiple partitions comprise two or more of: a chest partition, a neck partition, eye partitions, cheek partitions, a forehead partition, a nose partition, a mouth partition, and chin partitions.

19. The modular partitioned skin applicator of claim 10, wherein the multiple partitions comprise a digit portion and a joint portion.

20. A modular partitioned face mask, comprising:
multiple partitions shaped to fit a different portion of a user's facial topography, each of the multiple partitions having a skin-exfoliating adhesive coating; and
wherein the skin-exfoliating adhesive coating of each of the multiple partitions is of a strength corresponding to a portion of a user's facial topography or a characteristic of the user's epidermis.
